(19) United States
(12) Patent Application Publication
(10) Pub. No.: US 2020/0237846 A1
(43) Pub. Date: Jul. 30, 2020

(54) Cortex lycii tea formula

(71) Applicants: Hongping Tan, FLushing, NY (US); Boonteck Tan, FLushing, NY (US)

(72) Inventors: Hongping Tan, FLushing, NY (US); Boonteck Tan, FLushing, NY (US)

(21) Appl. No.: 16/260,502

(22) Filed: Jan. 29, 2019

(51) Int. Cl. A61K 36/815; A61K 36/481; A61K 36/488; A61K 36/287; A61K 9/00; A23F 3/34

(52) U.S. Cl. CPC A61K 36/815; A61K 36/481; A23F 3/34; A61K 36/287; A61K 9/0095; A61K 36/488

(57) ABSTRACT

This product is an herbal tea powder used to treat hyperglycemia (high blood glucose) and prediabetes. Relieving the main symptom of high blood glucose of type 2 diabetes. Research have shown that Cortex lycii (main component of the formula) can reduce blood sugar by stimulating pancreas β cell.

### BACKGROUND

Currently there are over 80 million Americans suffer from prediabetes and 30 Million from type 2 diabetes. Diabetes is one of the top 10 leading deaths in United States. When human intake glucose, it enters the bloodstream which triggers pancreas to secrete insulin. Insulin is a hormone that signal the cells that lack energy to accept glucose into its cell wall. Diabetic and prediabetes patients lacks the ability to generate enough insulin that the body is needed, which will show hyperglycemia symptom. When cells don't receive enough energy they will die off, which in long terms lead to organ failure. The most common efficient treatment currently is to inject patients with external insulin to balance out their glucose level.

### SUMMARY

This herbal tea formula is used to stimulate the pancreas β cell to produce adequate insulin to help maintain the glucose level in blood stream. When human body have balance glucose level they can protect their organ from failing due to lack of energy in cell. Insulin is an important hormone to maintain all human body cells function.

### SPECIFICATION

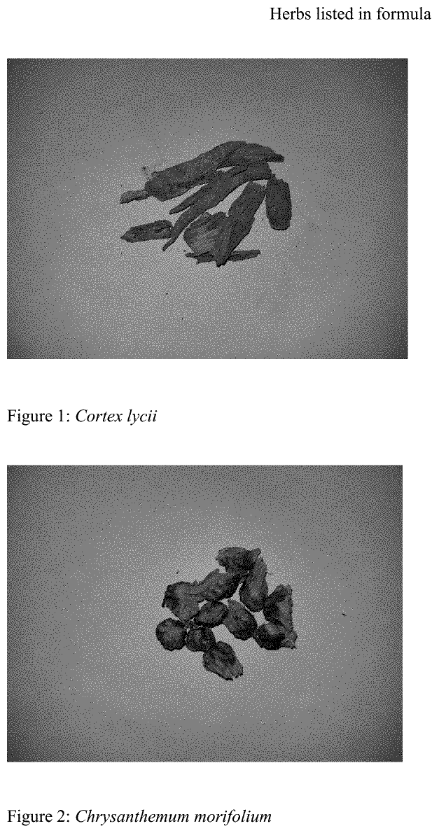

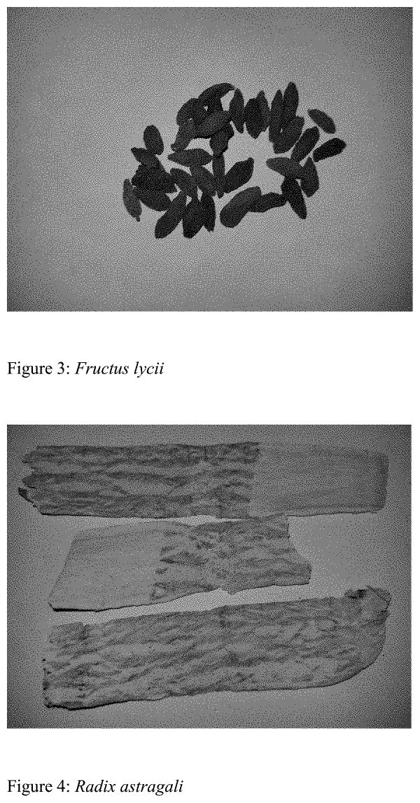

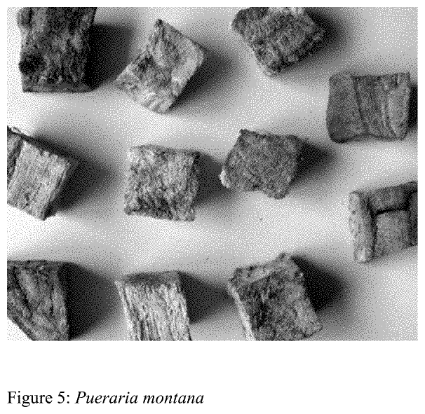

The Cortex lycii tea (net weight 15 g) contains 7.5 g of Cortex lycii (FIG. 1), 2.0 g of Radix astragali (FIG. 4), 2.0 g of Pueraria montana (FIG. 5), 2.0 g of Chrysanthemum morifolium (FIG. 2), 1.5 g of Fructus lycii (FIG. 3).

The FIG. 1 (Cortex lycii) shows dark brown sliced up dried roots from the plant Lycium barbarum.

The FIG. 2 (Chrysanthemum morifolium) shows the dried up dark orange flowers from the plant Chrysanthemum.

The FIG. 3 (Fructus lycii) shows the dried up red fruit from the plant Lycium barbarum.

The FIG. 4 (Radix astragali) shows the yellow-brown dried and sliced up pieces of root from the plant Astragalus propinquus.

The FIG. 5 (Pueraria montana) shows brown dried cube cut shape of the root from the plant Pueraria montana.

Each herb listed above will be in 70 mesh powder form. It will be contained in sealed aluminum bag. Every box will contain 30 aluminum bags. Per serving is 1 aluminum bag. The tea will be served with 250 m1 of water. Suggestion of consumption is 3 times a day. The inventor of this product is one of the researcher that published his finding on scientific journal. The paper can be found at https://www.omnicsonline.org/open-access/the-effect-of-cortex-lycii-on-rattus-norvegicus-blood-glucose-level-using-a-high-glucose-diet.pdf. This tea can prevent prediabetes from transforming into type 2 diabetes because it increases the production of insulin. The herbs have been used in traditional herbal medicine for other purposes and shown no side effects. This product can be consumed as a healthy beverage and should be available as OTC (over the counter).

The whole formula is created by both the researcher and an herbal medicine practitioner. The research mainly shows the effects of Cortex lycii, to better enhance the efficacy and create a desirable taste the formula was invented.

Production process of the product.

Each herb will be dry at moderate temperature using dryer. After the drying process all the herbs will be processed through crushing machine. The crushed herbs will then be grinded into 70 mesh. The powder will be mixed in a mixer machine with the proper proportion. Packaging machine will pack the mixed powder into aluminum bag.

1. A product for treating hyperglycemia comprising:
A powder form of Cortex lycii, Radix astragali, Pueraria montana, Chrysanthemum morifolium, and Fructus lycii.

2. The product according to claim 1, wherein the powder form mixture is consumed at 15 g of the powder into 250 ml of water.
